(19) United States
(12) Patent Application Publication
(10) Pub. No.: US 2007/0260216 A1
(43) Pub. Date: Nov. 8, 2007

(54) VASCULAR ACCESS CATHETER

(76) Inventors: Robert O. Hickman, Edmonds, WA (US); Marc Jaker, New Brighton, MN (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/675,281

(22) Filed: Feb. 15, 2007

Related U.S. Application Data
(60) Provisional application No. 60/774,003, filed on Feb. 15, 2006.

(51) Int. Cl. A61M 31/00

(52) U.S. Cl. 604/508

(57) ABSTRACT

An indwelling Fluoropolymer catheter primarily intended for vascular access. The catheter comprises a thin, lubricous sleeve circumferentially folded back over the leading edge of a pusher or positioning tube, affixed to a distal Hemostatic valve/guide assembly. The catheter may, in one embodiment, incorporate a needle or trocar for rapid deployment, which is loaded/positioned from the leading edge, back into the film membrane. The trocar may pierce the skin and advance through body tissues to an artery or vessel. The catheter is then inverted over the pusher tube following the guide of the trocar and optionally a guide wire into the artery. The trocar or needle will be totally withdrawn after catheter placement.

### CROSS-REFERENCE TO RELATED APPLICATIONS

This application claims priority to and benefit of U.S. Provisional Application No. 60/774,003, entitled “Vascular Access Catheter” and filed on Feb. 15, 2006.

### FIELD OF INVENTION

The present invention relates generally to vascular access catheters that are intended for long term use. In particular, the present invention relates to vascular access catheters such as those that may be used for dialysis, drug infusion, blood sampling, oncology, nephrology, or the administration of nutrition solutions.

### BACKGROUND OF THE INVENTION

Typical catheters are fabricated from silicone or urethane elastomer, and are very simple non-everting tubes. The importance of catheters has increased over the years and catheters now play a major role in acute and chronic care. In spite of the improvements made in catheter materials and related areas such as nursing care, catheter care, hospital and doctor office protocols, and with the bonding of drugs (e.g., antibiotics, anti-thrombotic agents), the incidence of infection and clotting episodes, when using catheters, is considerable. Many of the bouts of infection and thrombosis are life threatening or debilitating to the patient.

As an example of the increasing importance of catheters, approximately 12-15% of end stage renal failure patients will require central venous catheters. In addition, almost all oncology patients require central venous catheters as part of chemotherapy. Many other patients require central catheters for administration of potent and irritating drugs.

However, the incidence of infection and clotting episodes pose significant risks for the use of catheters, especially with patients who are already weakened by other causes. In addition, conventional central venous in-dwelling catheters may cause thrombosis due to their relative bulk. In particular, in smaller vessels, occluding caused by catheters will result in a restricted blood flow around the catheter, thus causing thrombosis. The present invention addresses these long felt needs with a new and improved catheter and method for using same.

### BRIEF DESCRIPTION THE DRAWINGS

A more complete understanding of the present invention may be derived by referring to the detailed description and claims when considered in connection with the drawing Figures, where like reference numbers refer to similar elements throughout the Figures, and:

FIG. 1 illustrates a catheter in accordance with an embodiment of the present invention;

FIG. 2 illustrates a valve and plug for use with a catheter in accordance with an embodiment of the present invention;

FIG. 3 illustrates a retention ring for use with a catheter in accordance with an embodiment of the present invention;

FIG. 4 illustrates a catheter and pusher tube in accordance with an embodiment of the present invention; and

FIG. 5 illustrates a catheter system in accordance with an embodiment of the present invention.

### DETAILED DESCRIPTION

The present invention may be described herein in terms of various hardware components and modules and processing steps. It should be appreciated that such modules and steps may be realized by any number of hardware components configured to perform the specified functions. For example, the present invention may employ various shaped tubes, sheaths, and the like, which may carry out a variety of functions. In addition, those skilled in the art will appreciate that the present invention may be practiced in any number of contexts and that the illustrative embodiment as described herein is merely one exemplary application for the invention. For example, the present invention may be applicable to various types of animals and other applications that require the use of various types of catheters. Further, such general techniques that may be known to those skilled in the art are not described in detail herein.

A catheter in accordance with the present invention proposes to reduce sepsis and infections through its everting design as described below. In addition, the catheter of the present invention is more patient friendly, as a needle is not required for re-entry.

A catheter in accordance with the present invention will permit rapid deployment, placement, and disposition of the catheter. It should be appreciated that such a catheter will be better tolerated than a conventional catheter. One reason for this is that a catheter in accordance with the present invention is non-occluding due to being constructed from “flat” tubing with a non-inflated total wall thickness in the vein of less than 0.002 inches. This is in contrast to conventional catheters that may have more than fifty times the wall section.

In addition, another aspect of the present invention is that the lumen may collapse flat when not in use. This is in contrast to conventional catheters that do not collapse flat when not in use. Often catheters that are in-dwelling, are not in use. Thus, when the catheter is not being used may have its lumen collapse flat in accordance with the present invention. In addition, when the lumen collapses, the backflow of blood is better prevented. This collapsing feature may also mean less blood or body fluids in the lumen which will result in less chance of infection, thrombosis, and sepsis. The collapsed profile will not restrict bloodflow as much as a conventional central venous catheter or alternatively create as much turbulence, which is associated with clotting through the vessel.

In accordance with an another embodiment of the present invention, a catheter in a “Trocar” system would allow for even more rapid deployment as such a catheter would puncture tissue, and the trocar system would be able to guide and deploy the catheter in one step. Only anchoring labor would somewhat mimic conventional cuffed catheters, but here too, the physical size of the catheter, the anchoring flaps, and the exit site would have lower profile than conventional catheters. Optionally, a catheter in accordance with the present invention could be deployed without use of a trocar. That is, the catheter could be deployed using conventional vascular-surgical techniques, at the discretion of a surgeon.

As the catheter is deployed, it everts as described below and likewise avoids the active transportation of bacteria in the blood.

The proximal end of the catheter may have a hemostatic valve to further control and restrict the backflow of fluids and blood primarily during a therapeutic infusion. In addition, the collapsed lay flat tubing serves to restrict backflow in a manner similar to a “duck-bill” valve. The hemostatic valve of the present invention may be specifically intended for polymer tubing, as opposed to stainless steel needles.

The length of the catheter from the anchoring flaps to the distal end, may range from 20-100 cm. The distal anchoring flaps may incorporate small, quick deployment butterfly expansion anchors. The anchors may then be enveloped in the surrounding body tissue. To later extract the catheter and its anchors, the whole catheter may be twisted several times, thus rolling the film body of the catheter into a tube shape, and thus drawing the anchors to the core catheter diameter from their expanded deployed position. The catheter may then be pulled straight out without requiring further manipulation or surgery.

The present invention provides a central venous access catheter that is made of a more biocompatible material than currently available. Its ongoing access is needle-less, thus better tolerated psychologically, minimizing venous scarring, and is less physically present, less invasive and therefore more comfortable than conventional venous catheters. Thus, a vascular access catheter in accordance with the present invention will likely be better tolerated for longer periods of time by the patient.

In accordance with an embodiment of the present invention, an indwelling Fluoropolymer catheter primarily intended for vascular access is provided. The catheter comprises a thin, lubricous sleeve circumferentially folded back over the leading edge of a pusher or positioning tube, affixed to a distal Hemostatic valve/guide assembly. The catheter may, in one embodiment, incorporate a needle or trocar for rapid deployment, which is loaded/positioned from the leading edge, back into the film membrane. The trocar may pierce the skin and advance through body tissues to an artery or vessel. The catheter is then inverted over the pusher tube following the guide of the trocar and optionally a guide wire into the artery. The trocar or needle will be totally withdrawn after catheter placement.

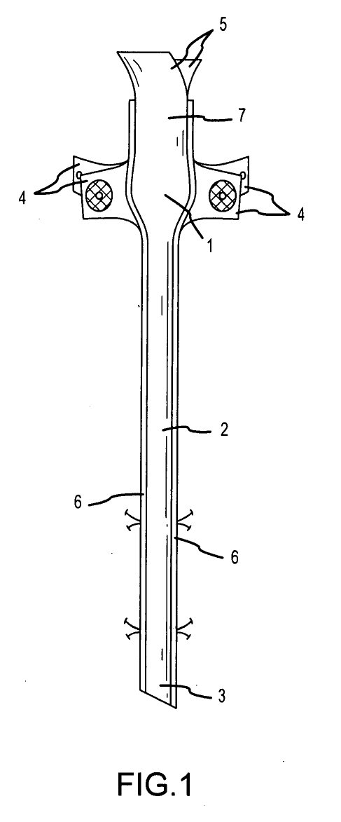

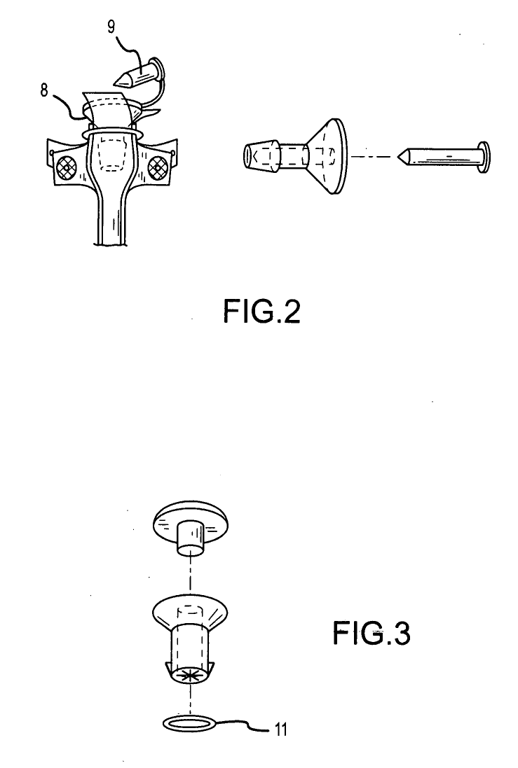

With reference to FIG. 1, an untensilized section 1 of a catheter in accordance with an embodiment of the present invention is illustrated. An outside area 7 of the catheter is illustrated, to which a hemostatic valve 8, as illustrated in FIG. 2, may be affixed. In accordance with an aspect of the present invention, hemostatic valve 8 may be affixed by a retention ring 11 illustrated in FIG. 3. In addition, plug 9 may be utilized, as illustrated in FIG. 2. Plug 9 may be attached to valve 8 by a tether or similar attachment.

With continued reference to FIG. 1, main body 2 of the catheter will invert and reside in the patient's tissue and vein and have the greatest blood contact. The catheter may be formed of two pieces of a fluoropolymer resin or fluoro-alloy such as polytetrafluoroethylene (PTFE) film. Such a catheter may have outside seams 6 as illustrated in the accompanying figures. Outside seams 6 may be formed by heat sealing the edges of the two pieces of film.

The Fluoropolymer catheter has a single or double external seams, over most of the length of the catheter. The fluoropolymer film may be tensilized and the external seams are generally small. In one embodiment, one section constituting approximately 1 inch or 2.5 cm, both seams near the proximal end plus additional untrimmed Fluoropolymer material form flaps at 90° to the axis of the catheter body. Anchoring flaps 4 may incorporate laser-machined holes in order to accommodate anchoring sutures or alternatively a bonded Polyester cuff. These flaps may sandwich a reinforcement structure (e.g., silk or cotton).

Anchoring flaps 4 may be sutured subcutaneously to the patient. Proximal alternate end flaps 5 may facilitate manipulation of the hemostatic valve. The anchoring may occur at or just below the skin. The extreme proximal end may also have flaps formed by cutting away the seams to accommodate manipulation of the entry valve portion of the catheter. These flaps may be parallel to the axis of the catheter body.

When the catheter is not in use, its lumen may collapse flat. It should be appreciated that when the lumen collapses, the backflow of blood or other fluids is better prevented.

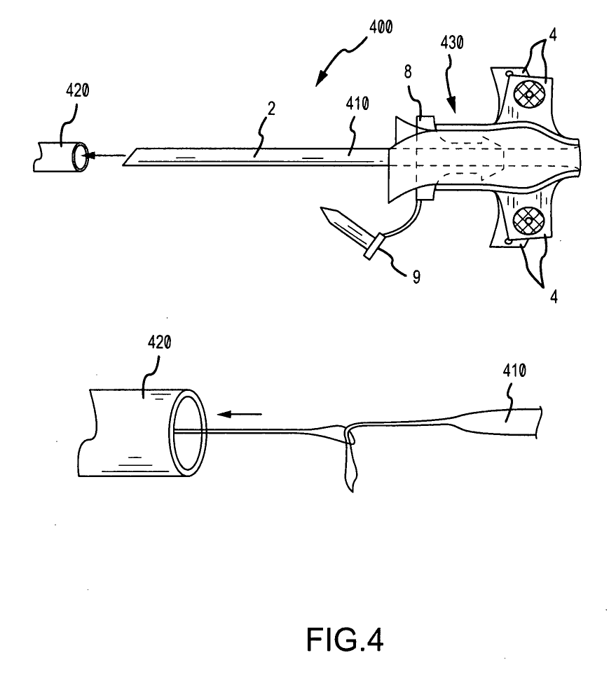

With reference to FIG. 4, a catheter assembly 400 is illustrated. Catheter assembly 400 comprises a catheter 410 with anchoring flaps 4, a pusher tube 420, and a hemostatic valve assembly 430. Valve assembly 430 comprises a hemostatic valve 8 and a plug 9. Plug 9 may be attached to valve 8 by a tether.

As shown in FIG. 4, catheter 410 is loaded into Polymeric pusher tube 420. The pusher tube may have an optional sharpened tip. This is optional because of the different types of procedures that may be carried out, and because of other factors such as anatomical placement, the performing surgeon's comfort level, and past experiences for these different applications.

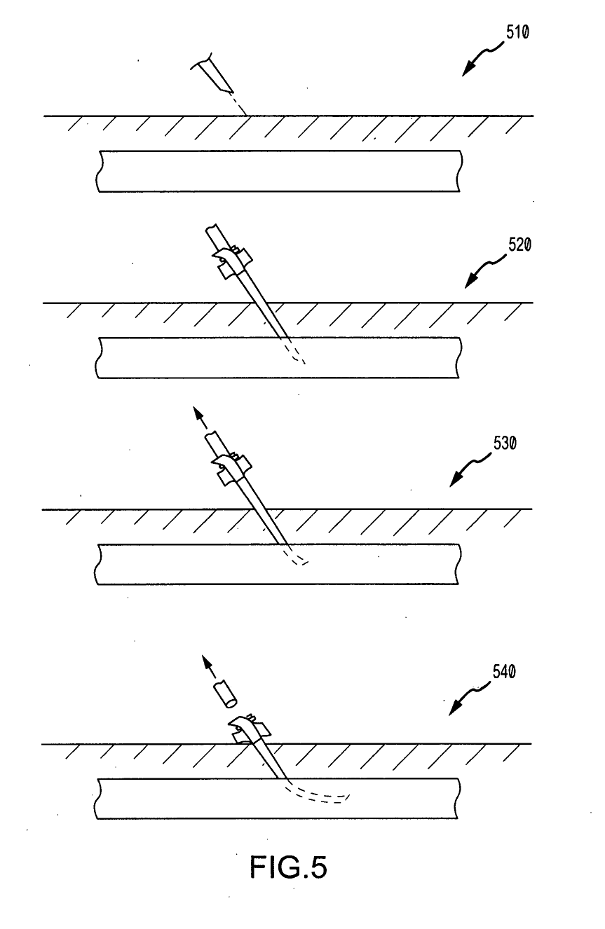

With reference to FIGS. 4 and 5, the main body 2 of the catheter will reside indwelling in the patient and is the section that is loaded into the inner diameter of the pusher tube. The surgeon or other user may puncture the vessel with a guide needle or the like (510). Section 2 of the catheter will evert into the patient through access made by the surgeon into the vein using a guide needle or similar device (520). After the catheter is placed, the pusher tube will be withdrawn and discarded (530). Flaps 4 may be anchored via suture to the patient and the patient's skin may be closed over (540). Plug 9 may be snapped into the Hemostatic valve 8 inner diameter and sealed. It is anticipated that a clear adhesive bandage/patch may be placed over the plug to further secure the exposed catheter.

In an alternate embodiment of the present invention, for perhaps even greater strain relief, the flaps could be pre-secured to a Dacron cuff that then in turn could be sutured to the patient's tissue.

A thin polymer film catheter, in accordance with an embodiment of the present invention, may comprise a film made from a fluropolymer, such as PTFE, or a similar performing material.

The present invention has been described above with reference to an exemplary embodiment. However, those skilled in the art will recognize that changes and modifications may be made to the exemplary embodiment without departing from the scope of the present invention. For example, the various processing steps dictated by the present invention, as well as the components for carrying out the processing steps, may be implemented in alternate ways depending upon the particular application or in consideration of any number of cost functions associated with the operation of the system. These and other changes or modifications are intended to be included within the scope of the present invention.

We claim:

1. A catheter for vascular access comprising:
a film membrane sleeve having a first end and a second end, wherein the film membrane comprises a fluoropolymer material; and
anchoring flaps formed from the film membrane sleeve, proximate to the second end.

2. The catheter of claim 1, wherein the film membrane sleeve comprises a first and second sheet of film.

3. The catheter of claim 2, wherein the first and second sheet of film comprise a polytetrafluoroethylene membrane.

4. The catheter of claim 3, wherein the first sheet of film is heat welded to the second sheet of film in a longitudinal fashion.

5. The catheter of claim 1, further comprising end flaps formed from the film membrane sleeve, wherein the end flaps are located between the anchoring flaps and the second end of the film membrane sleeve.

6. A catheter assembly for vascular access comprising:
a pusher tube having first and second open ends; and
a catheter comprising a film membrane sleeve having a first end and a second end, wherein the film membrane comprises a fluoropolymer material, and wherein the first end of the film membrane sleeve is disposed inside the tube.

7. The catheter assembly of claim 6, further comprising a valve assembly, wherein the valve assembly is connected to the second end of the film membrane sleeve.

8. The catheter assembly of claim 5, wherein the valve assembly comprises a hemostatic valve, a retention ring and a plug, wherein the hemostatic valve is connected to the second end of the film membrane sleeve by the retention ring.
